(19) United States
(12) Patent Application Publication
(10) Pub. No.: US 2021/0046184 A1
(43) Pub. Date: Feb. 18, 2021

(54) Process for Producing Water and Other Fluids with Increased Levels of a Measurable Life Force Energy by Placing the Fluids in the Proximity of Containers of Water with Existing High Levels of the Life Force Energy

(71) Applicant: William John Martin, South Pasadena, CA (US)

(72) Inventor: William John Martin, South Pasadena, CA (US)

(73) Assignee: MI Hope Inc, South Pasadena, CA (US)

(21) Appl. No.: 16/543,606

(22) Filed: Aug. 18, 2019

(51) Int. Cl. A61K 41/00; A61K 33/00; A61K 9/00

(52) U.S. Cl. CPC A61K 41/0004; A61K 9/0009; A61K 9/0004; A61K 33/00

(57) ABSTRACT

It is becoming increasingly recognized that water may provide health benefits beyond those attributed to hydration. The ability of water to provide such benefits can vary, however, with the original source of the water and how the water has been treated prior to it being consumed. A number of water-based products and water enhancing devices are currently being marketed with implied claims of providing significant health benefits to humans and animals. The beneficial water is variously referred to as being activated, energized, structured, micro-clustered, etc. The Applicant has explained the water activation process as the addition to the water of a natural energy force, which he has called KELEA (Kinetic Energy Limiting Electrostatic Attraction). He has developed sensitive assays for the detection and semi-quantification of KELEA in different manufactured water products. He has further shown that KELEA activated water can be utilized as a means of activating ordinary water by simply placing the ordinary water near to the KELEA activated water. This discovery will greatly expedite the widespread health, agriculture, and industrial uses of KELEA activated water.

### RELATED PUBLICATION BY THE APPLICANT

1. Martin, W. J. (2003) Stealth Virus Culture Pigments, A Potential Source of Cellular Energy. Experimental Molecular Pathology, 74, 210-223.
2. Martin, W. J. (2005) Alternative Cellular Energy Pigments from Bacteria of Stealth Virus Infected Individuals. Experimental Molecular Pathology, 78, 217-217.
3. Martin W J (2014). Stealth Adapted Viruses; Alternative Cellular Energy (ACE) & KELEA Activated Water. Author House, IN. pp 321. ISBN 978-1-4969-0496-6.
4. Martin, W. J. (2014) KELEA Activated Water—Enhancing the Alternative Cellular Energy (ACE) Pathway. In Stealth Adapted Viruses; Alternative Cellular Energy (ACE) & KELEA Activated Water. Author House, Bloomington, Ind., USA, pp. 115-144.
5. Martin, W. J. (2014) KELEA Activated Water Leading to Improved Quantity & Quality of Agricultural Crops. Advances in Plants & Agriculture Research, 2, 00033.
6. Martin, W. J. (2015) Therapeutic Potential of KELEA Activated Water. International J of Complementary & Alternative Medicine, 1(1), 00001.
7. Martin, W. J. (2015) Alternative Cellular Energy Pathway Therapy Using KELEA Activated Water. International J Complementary & Alternative Medicine, 2(2), 00051.
8. Martin, W. J. (2015) KELEA, A Natural Energy That Seemingly Reduces Intermolecular Hydrogen Bonding in Water and Other liquids. Open Journal of Biophysics, 5, 69-79.
9. Martin, W. J. and Laurent, D. (2015) Homeopathy as A Misnomer for Activation of the Alternative Cellular Energy Pathway, Evidence for the Therapeutic Benefits of Enercel in a Diverse Range of Clinical Illnesses. International J Complementary & Alternative Medicine, 2(1), 00045.
10. Dubrov, V., Dubrova, T., Christner, D., Laurent, D. and Martin, W. J. (2015) Alternative Cellular Energy Based Therapy Using Enercel® in Advanced AIDS Patients Co-infected with Tuberculosis and Treated in Chernigov, Ukraine. J Hum Virol Retrovirol., 2(6): 00061.
11. Martin, W. J. (2015) Interacting Light Paths Attract KELEA (Kinetic Energy Limiting Electrostatic Attraction) and Can Lead to the Activation of Water. Open Journal of Biophysics, 5, 115-121.
12. Martin, W. J. (2015) Interactive Electric Fields Can Attract KELEA (Kinetic Energy Limiting Electrostatic Attraction) and Can Lead to the Activation of Water. International J Complementary &Alternative Medicine, 1(6), 00034.
13. Martin, W. J. (2016) KELEA (Kinetic Energy Limiting Electrostatic Attraction) May Add to the Measured Weight of an Object. J Modern Physics, 7(6), 461-472.
14. Martin, W. J. (2016) KELEA (Kinetic Energy Limiting Electrostatic Attraction) Offers an Alternative Explanation to Existing Concepts Regarding Wave-Particle Duality, Cold Fusion and Superconductivity. J Modern Physics, 7(15), 1995-2007
15. Martin, W. J. (2016) Preparing and Using KELEA Activated Water to Enhance the Alternative Cellular Energy (ACE) Pathway in the Therapy of Multiple Illnesses. International J Complementary & Alternative Medicine, 3(1), 00059.
16. Martin, W. J. (2015) KELEA Activation of Water and Other Fluids for Health, Agriculture and Industry. Journal of Water Resource and Protection, 7, 1331-1344.
17. Martin, W. J. (2016) KELEA, Cosmic Rays, Cloud Formation and Electromagnetic Radiation, Electropollution as a Possible Explanation for Climate Change. Atmospheric and Climate Sciences, 6(2), 174-179.
18. Martin, W. J. (2016) Cancer as an Insufficiency of Cellular Energy (ICE), Therapeutic Approaches Based on Enhancing the Alternative Cellular Energy (ACE) Pathway. International J Complementary & Alternative Medicine, 3(3), 00074.
19. Martin, W. J. (2016) Insufficiency of Cellular Energy (ICE) in Neurons, From Electrical Hyperactivity to Quiescence. International Journal Complementary Alternative Medicine, 4, 00118.
20. Martin, W. J. (2016) Insufficiency of Cellular Energy (ICE) May Precede Neurodegeneration in Alzheimer's Disease and Be Treatable via the Alternative Cellular Energy (ACE) Pathway. Advances in Alzheimer's Disease, 6(1), 1-12.
21. Martin, W. J. (2016) Insufficiency of Cellular Energy (ICE), The Basis for Many Illnesses Potentially Correctable Using KELEA Activated Water. International J Complementary & Alternative Medicine, 4(1), 00106.
22. Martin, W. J. (2017) Cancer Is Treatable via the Alternative Cellular Energy (ACE) Pathway, Journal of Cancer Therapy, 8, 1279-1290
23. Martin, W. J. (2017) Using KELEA (Kinetic Energy Limiting Electrostatic Attraction) to Improve the Efficiency of Fuel Combustion. Open Journal Air Pollution, 6(3), 103-116.
24. Martin, W. J. (2017) Is KELEA (Kinetic Energy Limiting Electrostatic Attraction) a Source of Chemical Energy? MOJ Biorg Org Chem, 1(2), 54-58.
25. Martin, W. J. (2017) Insufficiency of Cellular Energy (ICE) from the Alternative Cellular Energy (ACE) Pathway Limiting the Specialized Functions of Neuronal Cells. International J Complementary & Alternative Medicine, 4(2), 00112.
26. Martin, W. J. (2017) Hyper-Excitability Followed by Functional Quiescence in Neuronal Cells Caused by Insufficient Cellular Energy (ICE), Treatable by Enhancing the Alternative Cellular Energy (ACE) Pathway. World Journal of Neuroscience, 7(3), 257-266.
27. Martin, W. J. (2017) Tissue Regeneration without Scarring Achieved by Enhancing the Alternative Cellular Energy (ACE) Pathway. Journal of Cosmetics, Dermatological Sciences and Applications, 7(1), 82-98.
28. Martin W J (2018) Is the placebo effect mediated by the Alternative Cellular Energy (ACE) pathway? International J Complementary & Alternative Medicine, 11(4): 231-233.
29. Martin W J (2018) KELEA Activated Water as an Alternative to Stem Cell Injections in Regenerative Medicine. International J Complementary & Alternative Medicine, 11(5), 251-258.
30. Martin W J (2019) Electromagnetic Radiation Causes Weight Loss and Weight Destabilization of Objects with Presumed Elevated Levels of KELEA (Kinetic Energy Limiting Electrostatic Attraction), Relevance to Human Health and to Global Warming. Modern J Physics 10(3), 195-213.

### RELATED PATENT APPLICATIONS BY THE APPLICANT

ACE-pigments and Humic acid as energy sources. Application Ser. No. 10/192,936
Method of assessing and of activating the alternative cellular energy (ACE) pathway in the therapy of diseases. William John Martin Submitted Jan. 16, 2008. Application Ser. No. 12/009,195
Enerceutical mediated activation of the alternative cellular energy (ACE) pathway in the therapy of diseases. Application Ser. No. 12/151,779
Regenerative wound healing using copper-silver citrate composition. Application Ser. No. 12/288,749
Enerceutical activation of the alternative cellular energy (ACE) pathway in therapy of diseases. Application Ser. No. 12/069,597
Method of using the body's alternative cellular energy pigments (ACE-pigments) in the therapy of diseases application Ser. No. 12/378,934
Urine as a source of alternative cellular energy pigments (ACE-pigments) in the assessment and therapy of diseases. Application Ser. No. 12/381,003
Activation of the alternative cellular energy (ACE) pathway in the therapy of diseases. Application Ser. No. 12/802,605
Methods for the detection of alternative cellular energy (ACE) pigments and for monitoring of the ACE pathway in the diagnosis and therapy of diseases. Application Ser. No. 12/802,763
Diagnostic value of systemic ACE pathway activation in the detection by fluorescence of localized pathological lesions. Application Ser. No. 12/804,619
Enerceutical mediated activation of the alternative cellular energy (ACE) pathway in the therapy of diseases. Application Ser. No. 12/151,779
Energy Charged Liquids to Enhance Enerceutical Activation of the Alternative Cellular Energy (ACE) Pathway in the Therapy of Diseases. Application Ser. No. 12/972,344
Energy Charged Alcoholic Beverages for Enhancing the Alternative Cellular Energy Pathway in the Prevention and Therapy of Diseases. Application Ser. No. 12/984,582
Energy Charged Liquids to Enhance Enerceutical Activation of the Alternative Cellular Energy (ACE) Pathway in the Therapy of Diseases application Ser. No. 12/972,344
Methods for Detecting and Monitoring the Activity of Energized Water and Other Liquids Useful for Enhancing the Alternative Cellular Energy Pathway in the Prevention and Therapy of Diseases. Application Ser. No. 13/016,948
Methods for Increasing the Kinetic Activity of Alcohol, Water and Other Liquids, so as to Render the Liquids More Useful in Enhancing the Alternative Cellular Energy Pathway in the Prevention and Therapy of Diseases. Application Ser. No. 13/029,116
Methods for Increasing the Kinetic Activity of Alcohol, Water and Other Liquids, so as to Render the Liquids More Capable of Enhancing the Alternative Cellular Energy Pathway in the Prevention and Therapy of Diseases application Ser. No. 13/040,262
Methods for Increasing the Kinetic Activity of Water and Other Liquids, so as to Render the Liquids More Useful in Enhancing the Alternative Cellular Energy Pathway and in Various Other Agricultural and Industrial Applications. Application Ser. No. 13/166,800
Use of Plants Extracts to Activate Water, Alcohol and Other Liquids. Submitted Oct. 27, 2011. Application Ser. No. 13/272,215.
Methods of Transferring Energies to Water, Alcohols and Minerals. Submitted Nov. 25, 2011. Application Ser. No. 13/304,558.
Use of Certain Foods and Dietary Supplements as Water and Beverage Activating Enerceuticals. Application Ser. No. 14/507,822
Heat as a Method to Enhance the Fluid Activating Ability of Humic Acids, Zeolites and related Enerceuticals. Application Ser. No. 14/294,076
Method of Enhancing the Alternative Cellular Energy Pathway in Humans and Animals Using Wearable Items That Contain KELEA Activated Water. Submitted Feb. 18, 2019. Application Ser. No. 16/278,712
Method of Detection and Measurement of a Life Force Energy, Also Known as KELEA. Submitted Feb. 27, 2019. Application number not currently available.
Method of Detection and Measurement of a Life Force Energy, Also Known as KELEA in Humans, Animals, Plants, Equipment, and the Environment. Application Ser. No. 16/508,255

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### FIELD OF THE INVENTION

The invention is within the field of a newly defined form of energy, which has major health, agricultural and industrial applications. The energy is referred to by the Applicant as KELEA, being an abbreviation for kinetic energy limiting electrostatic attraction KELEA is comparable to what has been described in traditional oriental medicine as the universal life force. Other terms applicable to KELEA include chi, prana, mana, vital energy, orgone, etc. It is also commonly implied in traditional medical teachings that certain specifically located sources of water can provide this life force energy when consumed by humans. Some individuals further claim that it is possible to transform ordinary or regular water into water with additional life enhancing properties. Terms used to describe such water include activated, energized, structured, micro-clustered, living, and revitalized. Many of these water products are commercially available but without any Food and Drug Administration (FDA) acknowledgement of proven medical value. The methods used to produce these commercially available water products are generally not fully disclosed; nor is the basic mechanism of activation generally understood. Moreover, promoters of these products commonly further argue that their particular product has unique beneficial properties that are not shared with their competitors' products. There have been occasional suggestions that activated water may be able to indirectly contribute to the beneficial properties of additional water without the need for direct contact. The closest suggestion was the work of Johan Grander (www.grander.com). Although not publicly disclosed, his original water activating device contained an inner chamber with fluid from an Austrian mine that was supposedly rich in copper. Water would enter into and exit from an outer compartment in his device. Others have argued that even passing water through devices that establish turbulence can lead to the activation of the water. The major barriers to progress in this field of endeavor has been not understanding how water becomes activated and not having a simple assay to detect activation.

Through his original research, the Applicant has been able to clarify many of the issues relating to the production and usage of life enhancing activated water. This research is presented in a series of prior patent applications and publications, which are incorporated herein by reference. As noted above, the research has led to the identification of KELEA as an energy form that be carried by water and other fluids. The present invention describes the use of KELEA activated water as a continuing source of KELEA, which can be utilized to transfer KELEA into nearby water. This discovery was aided by the development of suitable assays to determine the approximate level of KELEA activation of water. The invention will greatly increase the availability of activated water for its many health, agricultural, and industrial uses.

### BACKGROUND OF THE INVENTION

The value of an adequate intake of water is unquestioned in medicine and in agriculture. It is also widely accepted that certain sources of water have added benefits over what is regarded as regular or ordinary water. The majority opinion is, however, that the added benefits of the water from these sources are due to either the presence of minerals or the absence of toxic components in the water. An alternative belief is that the structure of water from certain sources is intrinsically different from the structure of the less beneficial water. Speculations regarding the structural differences have included smaller sized groupings of more tightly connected water molecules (micro-clustered water); organization of the water in a manner that reflects or memorizes the changes caused by prior content of a beneficial compound, as implied in homeopathy; wider spacing of the hydrogen atoms in individual water molecules, etc.

The Applicant use of the term activated is intended to be a generic term for water that has enhanced biological and other energy-based properties, when compared to ordinary or regular water; which are due to an intrinsic change in the water and not due to the presence or absence of additional compounds in the water. The term activated is, therefore, synonymous with the previously listed terms for beneficial water.

Based on extensive research, the Applicant has proposed that activated water has an added dynamic or kinetic quality and is reflected in a loosening of the hydrogen bonding between water molecules. He attributes this to the absorption of an external force, which he terms KELEA, an abbreviation for Kinetic Energy Limiting Electrostatic Attraction. The fundamental role of KELEA in Nature is thought to prevent the fusion and elimination of adjacent electrostatically attracted opposite electrical charges. Various dipolar compounds, that is compounds with separated positive and negative electrical charges, can attract KELEA. When added to water, some of these compounds can transfer KELEA into the water, possibly in an oscillatory and continuing manner. Water activating dipolar compounds include certain preparations of the following: humic and fulvic acids, zeolites, mineral oxides, mica, powdered and heated volcanic rocks, various herbal tinctures, lidocaine, and many others.

The Applicant's research on activated water led to the development of several novel assays. These have included 1). Increased volatility as measured by the increased rate of weight loss in capped but not completely sealed containers of the activated water, when compared to similar capped containers of regular water. The increased volatility can also be seen in the progressive expansion of heat-sealed Ziploc bags containing KELEA activated fluids. 2). The dissolving pattern of sprinkled particles of neutral red dye, as best observed using a microscope and small circular dishes. The patterns can range from stationary particles remaining on the surface of water, which slowly dissolve to yield expanding circular red discs of the dye, to the following changes, which are semi-quantitative characteristics of activated water: a) The particles of neutral red dye readily break through the surface of the water indicating a reduced surface tension of the water; b) the particles show relatively rapid linear back and forth movements of up to a centimeter; c) residual small particles of undissolved dye will cluster into a group from which individual particles are occasionally rapidly rejected, only to slowly return back towards the cluster, usually to be expelled again; d) ultraviolet (UV) fluorescence of the solution of dissolved neutral red dye. 3). Formation of a vertical vortex within the water in a container that is spun by hand for a few seconds. The vortex can last for over 30 seconds. 4). The behavior of lidocaine crystal particles when a small number of the particles are sprinkled onto the water. Lidocaine powder was obtained from Sigma-Aldrich Life Sciences. It is also widely available from other sources. Lidocaine powder is poorly soluble in room-temperature water. The relatively larger crystal particles of lidocaine powder show only minimal movements when a few of the particles are sprinkled onto the surface of inactive water. They will, however, display dramatic movements, including vigorous rotations, linear motions, and repeated attachments and dis-attachments from each other and from any additional microscopic particles, when sprinkled onto highly activated water. The rapid dis-attachments are viewed as a direct manifestation of KELEA. The jerky movements can continue beyond a minute and can usually be reestablished by repeated jolting of the dish or other container against a hard surface. Less intense movements of the lidocaine particles, which range between the two extremes will occur with water samples that have lower levels of activation. With experience, this assay can provide a reliable semi-quantitative estimation of the level of water activation. The assay is currently performed on water samples placed into 1.25″ diameter, 0.75″ high circular dishes (BoxBox Brand purchased the Container Store, Pasadena, Calif.). The movements of the particles are viewed directly and with a low magnification microscope. Lidocaine crystals are phosphorescent and can also be seen as bright objects against a black background when not using the light from a microscope. Microscopically, the particles added to Enercel show very rapid electrostatic attachment and dis-attachment with one another. Tap water is commonly used as a negative control. The lidocaine assay has yet to be publicly disclosed. Certain other floating dipolar particulate materials, which also show more increased attachment and dis-attachment and other movement activities in activated water compared to regular water have been identified and include a poorly soluble humic acids powder from Morningstar Minerals, NM. 5). The ability to add several milligrams to the measured weight of a single rolled sheet of paper that is placed close to a plastic container of the water. 6). Its interaction with humic acid particles such that small bubbles of gas (vaporized water) will gradually form around the particle before collapsing. Using combinations of these various assays, the Applicant has been able to readily discern the approximate levels of KELEA activation of water samples.

### BRIEF SUMMARY OF THE INVENTION

Using one or more of the above assays for activated water, the Inventor has repeatedly shown that what was originally regular or non-activated water can be transformed into highly activated water by merely being stored for periods of time in close vicinity to a collection of previously activated water. The secondarily activated water can then be used to continue the process of activating additional water. In other words, there is no limit to the ability to continue to transform regular water to activated water. The activation process is attributed to the continuing transfer of KELEA from the activated water to regular water.

### BRIEF DESCRIPTION OF THE DRAWINGS

Not Applicable and none included

### DETAILED DESCRIPTION OF THE INVENTION

The Applicant has focused these studies on three available sources of activated water, as well on water, which the Applicant has directly activated. The outside sources of water are called Enercel, VEW, and EES. Enercel is manufactured by World Health Advanced Technologies Inc. in Sarasota, Fla. (www.enercel.com). It is a diluted mixture of different herbal tinctures. The formula is essentially similar to that of a product from Argentina called HANSI, an abbreviation for homeopathic activator of the natural system immune. Based on a 1992 communication from the Applicant to the US manufacturer of Enercel, the name of the US made product was changed from HANSI to Enercel. It has been proven to have outstanding clinical value when administered intramuscularly and/or intravenously to patients with serious medical diseases, including tropical diarrhea, AIDS, tuberculosis, hepatitis, and asthma (please refer to references 4, 9, 10 in the list of published articles by the Applicant). It also prevents scar tissue developing in a burn (reference 27). Modified forms of Enercel are available as a skin spray and as an inhalant. Two hundred ml of Enercel for intravenous administration was added to a polyvinyl chloride (PVC) container that was sealed by heat. This product has been used clinically as a “waterceutical pouch.” It was also used to activate regular water that was placed neat to the pouch.

VEW stands for Vortically Enhanced Water (www.starchamberproducts.com). It is prepared from deionized water in a 200-gallon metal tank located in a water bottling facility in California. The source of the deionized water is from an Air Force base. A proprietary mixture of minerals is applied to the outside of the metal tank for 24 hours, before the water is bottled.

EES refers to Energy Enhanced System (www.eesystem.com). It consists of inwardly facing sets of opposing computers, which continually display scrolling patterns of lines of letter shaped symbols. The computers are arranged such that the images from the computers converged to a central area in the room containing the computers. Bottled water becomes activated when left in the room for more than a day. A system operating in North Carolina that is located close to a water bottling plant was used to activate several cases of bottled water obtained from the water bottling plant. These cases of EES-activated water were subsequently shipped to the Applicant. The Applicant separately received a shipment of several cases of bottled water directly from the water bottling plant. The water in this shipment was stored separately from the EES activated water till the time of testing.

The Applicant has also variously prepared several gallons of KELEA activated water. An early approach was to use calcined magnesium oxide (MgO) pellets (CropMag 200, obtained from Martin Marietta). This material at approximately 1 gram per 10 ml has been in contact with water for over 6 years. As the water is partially used for different purposes, it is simply replenished. Another approach has been to use humic acid (e.g. Black Silk Powder from Necternal Labs. Idaho, www.necternal.com). Point one percent (0.1%) of the humic acid is added to store purchased distilled water, with occasional repeated (e.g. ten-twenty times) succession (jolting) of the container against a hard surface, 4-6 times over a 24-hour period. Two subsequent 10-fold dilutions are made into distilled water at 24-hour intervals, with periodic jolting of the container. This is followed by zero-residue filtration of the fluid to remove all of the added humic acid. The distilled water being used is also sometimes first passed through a zero-residue filter. A third current approach to activating water has been to bubble Brown's gas produced by an acrylic polishing machine (HHO flame generator manufactured in China) into a liter of distilled water for several hours. The water is subsequently neutralized with hydrochloric acid and passed through a zero-residue filter. The fourth current approach to activating water is to electrolyze silver, followed by copper, in a citric acid—potassium chloride solution, as described in patent application Ser. No. 12/288,749. The copper-silver-citrate (CSC) solution has been shown to expedite the healing of wounds (reference 27).

The water provided from the above-mentioned sources (Enercel, VEW, and EES) and locally produced water (MgO, humic acid, and CSC) have all been confirmed as being KELEA activated using various combinations of the assays methods previously discussed. Studies have been performed to test whether each of these products could be used to increase the KELEA activity in regular water. For this purpose, drinking and distilled water in one-gallon containers were purchased from a local grocery store. Bottle water of different brands have also been purchased as single bottles. Regular tap water has been obtained from multiple sources. Five gallons of deionized water were obtained from the California Institute of Technology.

Containers of water in which the water was assessed using several of the different assays for measuring KELEA activation as having a low level of preexisting activity were positioned close to the different supplies of KELEA activated water. The introduced water was subsequently reassessed at different times using the same assays as were used on the water prior to it being brought into the room with the KELEA activated water. Without exception, the placement of regular water in the vicinity of KELEA activated water has resulted in the newly introduced water becoming more active. The activation was regularly noted at 24 hours, although it clearly began sooner. After several days, the activation was such that both the originally activated and the secondarily activated water showed comparable activity.

For example, a gallon of regular distilled water was brought into a room containing a single case of twenty-four 350-ml bottles of VEW as the energy source water. The case of VEW was then jolted 20 times by repeatedly lifting it about a foot and dropping it to the floor. This process leads to added activation of the source water. The energy receiving distilled water was also occasionally jolted. When examined at 24 hours, the distilled water showed significantly increased activities in both the neutral red dye and the lidocaine-based assays. Indeed, it was considered to have become essentially comparable in activity as the VEW product. The lidocaine assay was the easiest assay to approximately quantify the comparable levels of KELEA activity of the energy source and energy receiving water.

A similar study was performed in a separate room. This room contained a case of the EES activated bottled water and two cases of regular water from the same water bottling plant that had supplied the water for EES activation. These two cases had been separately shipped to the Applicant and were maintained in separate location until the initial testing was completed. Several additional gallons of regular water have also been stored near the EES-activated water. Again, the storage of the regular water in the room with the EES-activated water led to it becoming more active in both the neutral red dye and the lidocaine assay. The indirectly activated water also showed a prolonged vertical vortex spinning time when lightly spun.

A succussed PVC container of 200 ml of Enercel was shown to increase the volatility of adjacent vials of regular distilled water. The flexible PVC container was also tested by placing it within a gallon container of distilled water. This too led to the secondary activation of the water. Using both approaches did not lead to any apparent diminution of the measured activity of the Enercel when it was subsequently retested in the neutral red dye and lidocaine KELEA assays.

Supplies of activated water prepared by the Applicant using the three methods described above (MgO, humic acid, and CSC), have each shown similar non-contact activation of regular distilled and drinking water. A significant degree of activation occurs with tap water, although tap water is seemingly less efficiently activated than distilled water. Activation was especially efficient using water from the five-gallon container of deionized water. Plastic bottles and PVC containers were used throughout the studies.

The secondarily activated deionized water has now been used to repeatedly activate several gallon containers of regular distilled water. The water in these treated containers has shown activity that is at least as good as the VEW and EES activated water and nearly the same as that of Enercel. The most highly activated water is that which was previously produced by the Applicant using a Van de Graaff generator to electrostatically activate steam.

The neutral red and lidocaine assays have proven to be extremely useful in measuring the activity of both the KELEA source and the KELEA receiving water. For added reassurance, many of the activated samples of water have now also been tested for their volatility, vortex formation, and ability to add to the weight of a rolled sheet of typing paper. The sources of activated water have been in continual use for over two-months without apparent decay in their levels of activity. One provision is the need for tightly capping containers of the activated water if they are stored away from other sources of activated water. This is because of the heightened volatility of the more active water molecules.

An incidental observation made in these studies was that on several occasions the water in some store-purchased containers have shown a much higher than expected KELEA activity level. Multiple brands of water were present in the store. Arguably, only one of the brands needed to be KELEA activated for it to activate all of the remaining water brands.

There is no upper limit to the potential amount of water, which can be activated using a core sample of water with a high level of KELEA. This is because the secondarily activated water can proceed to activate additionally placed water. Based on experience, a reasonable starting ratio of the volume of the water to be activated to the volume of the water with a preexisting heightened level of KELEA that is being used is approximately ten-fold. The time for activation should normally exceed 24 hours with 3-6 periodic jolting of the activating water.

Wearable waterceutical pouches have been provided to individuals for their clinical evaluation. In a recent shipment, some of the pouches contained VEW, while other pouches contained water from the secondary activated 5-gallon deionized water. The Applicant contends that the different pouches will have comparable beneficial clinical activity.

The findings reported herein can greatly expand upon the widespread availability of KELEA activated water. An immediate application is the decision to add a core grouping of cases of EES-activated water into the main bottled-water storage facility at North Carolina. As the surrounding cases of water become activated, they will be expected to lead to the activation of the next further placed cases of water in a continuing time dependent process. The speed of widespread activation can be increased by the daily jolting of the core grouping of cases. The lidocaine motility assay can be used to monitor the progressive water activation process. Similar approaches are planned for use in other water bottling plants and bottled water storing facilities. Containers of water activated by any of several different methods can also be included in the tanks of water awaiting to be bottled or provided for other usages, including in the preparation of beverages and many water-containing packaged-food products. Activated water can greatly improve the quantity and quality of agricultural crops as well improving lawns and domestic gardens. Containers of activated water that may need to be periodically jolted can be attached to water tanks or to water lines providing irrigation of crops. Attaching a container of activated water to a household or building water supply can improve the efficiency of heating the water and preventing scale formation and corrosion of metal pipes. Swimming in pools with activated water should be beneficial. Multiple additional ways can be envisioned to apply the principles of the discovery described in this application. Progress will be greatly facilitated by routinely using the described KELEA activation assessment assays, especially the lidocaine mobility assay.

The potential medical uses of activated water are enormous. The Applicant is pioneering the concept of Regenerative Energy Assisted Clinical Healing (REACH)™. Progress will be limited if the availability of activated water depends on the poorly funded limited current suppliers of products such as Enercel and VEW. Their products as well as other sources of KELEA activated water can now be used as a beginning catalyst to create water products with comparable activity. One example in medicine is the activation of saline solutions widely used in medical facilities. Activated water needs not to be administered parenterally and benefits can be seen using KELEA activated water in pouches applied to the skin. It can also be used as a skin spray and as an inhalant.

The non-contact or indirect secondary activation process can also be used with other fluids in a reciprocal manner. The other fluids can include beverages; liquid based food products, such as soups; alcohol; gasoline; and diesel fuel. Humic acid activated alcohol can be used as starting material to activate water. The lidocaine assay is not suitable for use with alcohol because it dissolves too readily. The neutral red dye assay is not especially useful in alcohol because it shows a relatively high level of background activity even prior to it being activated. For lidocaine testing of certain beverages and liquid based foods, it is necessary to first extract and purify their water component.

A major reason for promoting the use of KELEA activated water is the evidence obtained by the Applicant that the natural availability of KELEA as a natural life force energy has been reduced by manmade electromagnetic radiation (Reference article number 30). It is extremely fortunate, therefore, that the described method is available to allow for unlimited supplies of KELEA activated water. Various types of containers that are able to transmit KELEA to nearby water can be developed for various health, agriculture and industrial applications. Providing small quantities of lidocaine, neutral red dye, and other substances will allow for the easy assessment of KELEA activity of water from different sources.

1. A method for producing fluids that have increased activity in assays for a beneficial energy force that is referred to by the Applicant as KELEA, comprising the placing of the fluids to be activated in the vicinity of water, which has a desired, heightened level of KELEA activity, for a sufficient period of time so that the KELEA activity of the newly introduced fluids increases to that of the water that is used in the water activating process.

2. The use of water that has been activated by being placed in the vicinity of a water with a high level of KELEA activity as a substitute and/or a replacement for the many known beneficial uses of water in which with a heightened level of KELEA activity has been created using other means of water activation.

3. A method of assaying the KELEA activity of water comprising the sprinkling of a few particles of lidocaine powder onto the surface of the water and observing the particles for their movements across the surface of the water and for repetitive attachments and dis-attachments of particles from one another, in which the extent of the movements and the attachments and dis-attachments are directly correlated with the KELEA activity of the water.

4. The method of claim 1 in which the fluid to be activated by being placed in the vicinity of water, which has a desired, heightened level of KELEA activity, is water.

5. The method of claim 1 in which the fluids to be activated by being placed in the vicinity of water, which has a desired, heightened level of KELEA activity, are beverages.

6. The method of claim 1 in which the fluids to be activated by being placed in the vicinity of water, which has a desired, heightened level of KELEA activity, are liquid food products.

7. The method of claim 1 in which the fluids to be activated by being placed in the vicinity of water, which has a desired, heightened level of KELEA activity, are pharmaceutical products, such as normal saline and dextrose solutions for intravenous administration.

8. The method of claim 1 in which the fluid to be activated by being placed in the vicinity of water, which has a desired, heightened level of KELEA activity, is water that is intended to be consumed as drinking water.

9. The method of claim 1 in which the fluid to be activated by being placed in the vicinity of water, which has a desired, heightened level of KELEA activity, is water that is intended to be used for bathing or for swimming.

10. The method of claim 1 in which the fluid to be activated by being placed in the vicinity of water, which has a desired, heightened level of KELEA activity, is water that is intended to be provided to animals.

11. The method of claim 1 in which the fluid to be activated by being placed in the vicinity of water, which has a desired, heightened level of KELEA activity, is water that is intended to be used for agricultural and/or gardening purposes.

12. The method of claim 1 in which the activating water is placed adjacent to the fluid that is intended to be activated.

13. The method of claim 1 in which the activating water is placed into a container that is submerged into the fluid that is intended to be activated.

14. The method of claim 1 in which the period of time that the fluid to be activated is placed in the vicinity of water with a heightened level of KELEA activity is from 24 to 72 hours.

15. The method of claim 1 in which the volume of fluid to be activated is in excess of ten times the volume of the KELEA activated water that is used in the fluid activation process.

16. The method of claim 1 in which the level of KELEA activity in the water being used to activate other fluids is further increased by periodically jolting the containers of the water at intervals of 4-6 times over each 24-hour period.

17. The method of claim 3 in which the assay using lidocaine powder is used to determine the level of KELEA activation of naturally available untreated water.

18. The method of claim 3 in which the assay using lidocaine powder is used to determine the efficiency and effectiveness of different methods that are intended to increase the KELEA level of water.

19. The method of claim 3 in which particles of compounds that show comparable movement activities as does lidocaine when sprinkled onto KELEA activated water are used as an alternative to the use of lidocaine in the assay for KELEA activity of a fluid.
